(19) United States
(12) Patent Application Publication
(10) Pub. No.: US 2024/0331851 A1
(43) Pub. Date: Oct. 3, 2024

(54) UNREGULATED DEVICE USAGE IDENTIFICATION AND TRACKING

(71) Applicant: International Business Machines Corporation, Armonk, NY (US)

(72) Inventors: Paul R. Bastide, Ashland, MA (US); Matthew E. Broomhall, Goffstown, NH (US); Robert E. Loredo, North Miami Beach, FL (US)

(21) Appl. No.: 18/191,428

(22) Filed: Mar. 28, 2023

(51) Int. Cl. G16H 40/20; G16H 10/60

(52) U.S. Cl. CPC G16H 40/20; G16H 10/60

(57) ABSTRACT

Tracking unregulated device usage in a clinical setting is provided. A patient caseload for a medical device is determined via electronic health record (EHR) data for the clinical setting. A capacity for the medical device in the clinical setting is determined by accessing an electronic inventory. Overcapacity of the medical device is determined based on the capacity and the patient caseload. An alert on unregulated device usage is generated in response to determining the overcapacity of the medical device.

### BACKGROUND

The present disclosure relates to management of devices in a clinical setting, and more specifically, to management of unregulated devices in a medical setting.

In the United States, the U.S. Food and Drug Administration regulates medical devices to assure their safety and effectiveness. However, in certain emergency situations, the need arises for unregulated device usage to fill a gap in available medical devices. For example, during the COVID-19 pandemic, hospitals were authorized to use unregulated ventilator splitters to increase the number of patients that their existing ventilators could treat.

### SUMMARY

According to embodiments of the present disclosure, a computer-implemented method for tracking unregulated device usage in a clinical setting is provided. The method includes determining a patient caseload for a medical device via electronic health record (EHR) data for the clinical setting. The method further includes determining, by accessing an electronic inventory, a capacity for the medical device in the clinical setting. The method further includes determining overcapacity of the medical device based on the capacity and the patient caseload. The method further includes generating an alert unregulated device usage in response to determining the overcapacity of the medical device.

According to further embodiments of the present disclosure, a computer program product and system for performing the method are provided.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a block diagram of example computing environment, according to embodiments of the present disclosure.

FIG. 2 depicts a block diagram of an unregulated device usage module, according to embodiments of the present disclosure.

FIG. 3 depicts a flow diagram of an example computer-implemented method for managing unregulated device usage, according to embodiments of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to management of devices in a clinical setting, and more particular aspects relate to identifying unregulated device usage and tracking unregulated device usage in a clinical setting. While the present disclosure is not necessarily limited to such applications, various aspects of the disclosure may be appreciated through a discussion of various examples using this context.

As used herein, unregulated device usage refers to the use of devices that are not approved for medical use or devices that are being used in an unapproved way. In the United States, the Food and Drug Administration (FDA) regulates medical devices. During an emergency situation, the FDA may authorize unregulated medical devices or unregulated uses of approved medical devices to be used in an emergency to diagnose, treat, or prevent serious or life-threatening diseases or conditions when certain criteria are met, including there are no adequate, approved, and available alternatives. As used herein, a device may be a mechanical device, electronic device, or a combination thereof.

Although unregulated device usage may be proper in a clinical setting in certain emergency situations, it is difficult to identify and track their usage. Thus, it may be difficult to stop the unregulated device usage when the emergency situation has ended or provide scheduled maintenance for these devices. There is a need to better manage the lifecycle of unregulated device usage.

Existing electronic health record (EHR) systems are not designed to record unregulated device usage. Some EHR systems allow for a medical device to only be assigned to a single patient at a time because the medical device is only approved for use with a single patient. However, while the medical device is only approved for use with a single patient, it may be necessary, in an emergency situation, to use the device in an unapproved way that allows it to be used with multiple patients. Further, using the device in an unapproved way may involve using additional devices. For example, during the COVID-19 pandemic, splitters were used to allow a single ventilator to be used with more than one patient. The design of some EHR systems implement rules that do not allow for a medical professional to assign a unique ventilator to more than one patient when more than one patient is using the ventilator. Additionally, EHR systems may only allow for assigning approved medical devices to a patient. Thus, unregulated devices may not be clearly assigned to a patient, such as devices created in situ in response to a shortage of approved devices.

Identifying and tracking the unregulated device use in a clinical setting may allow for improved management of unregulated device use. For example, tracking an unregulated device may allow for the device to be identified for use with another patient when it is no longer being used. Additionally, when the unregulated use is no longer necessary, the unregulated usage can be stopped and replaced, if necessary, with approved device usage. Further, the tracking of unregulated devices may allow for generating a maintenance schedule for the unregulated devices.

Embodiments of the present disclosure may provide a method, system, and computer program product for identifying the use of unregulated devices in a clinical setting. The patient caseload for a medical device may be determined by accessing electronic health record (EHR) data. The patient capacity of the medical device within the clinical setting may be determined. Overcapacity of the medical device may be determined by comparing the patient caseload to the patient capacity of the medical device. In response to determining overcapacity of the medical device, an alert may be generated.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as unregulated device usage module 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

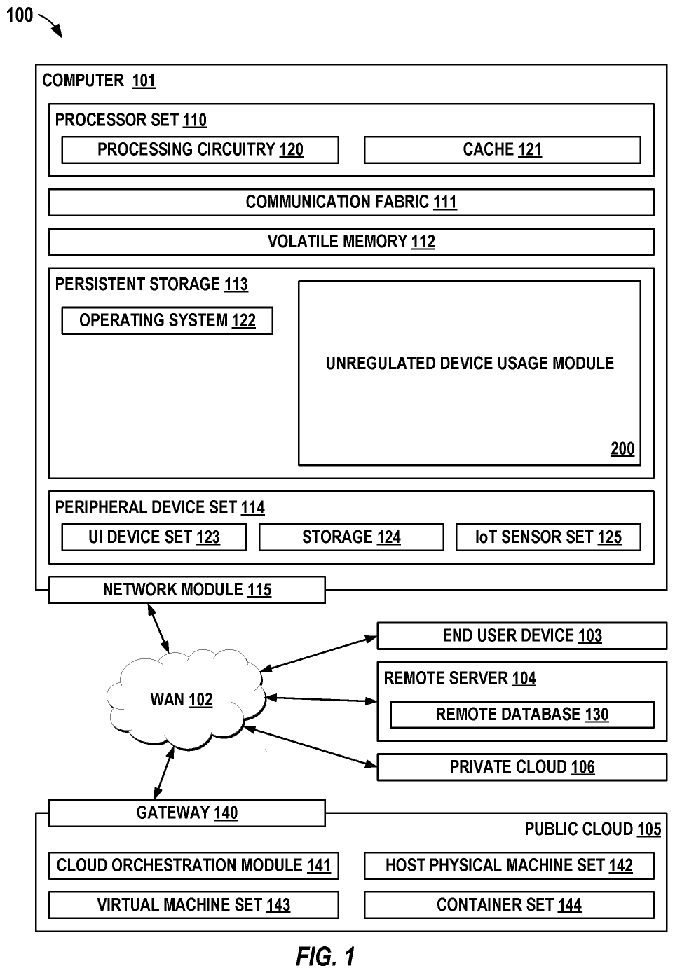

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

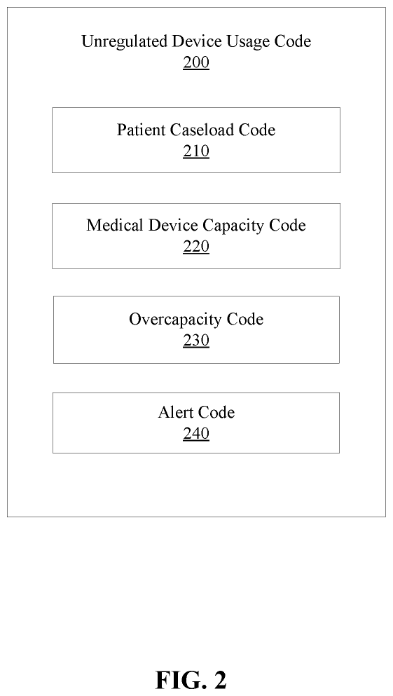

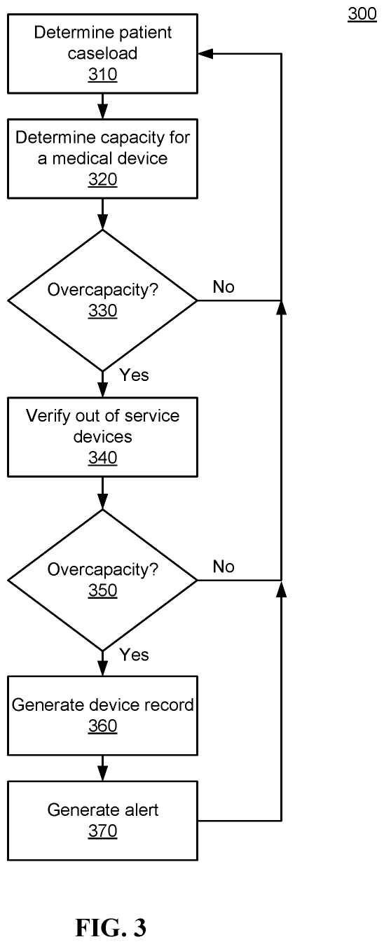

Referring now to FIG. 2, a block diagram of unregulated device usage module 200 is depicted, according to embodiments. As depicted in FIG. 1, unregulated device usage module 200 may contain code executable by computer 101 to cause computer 101 to perform one or more methods such as method 300 described in reference to FIG. 3. Unregulated device usage module 200 includes patient caseload code 210, medical device capacity code 220, overcapacity code 230, and alert code 240.

Patient caseload code 210 may contain code for determining a patient caseload for a medical device using EHR data. For example, patient caseload code 210 may contain code configured to cause computer 101 to perform operation 310 described in reference to FIG. 3. Medical device capacity code 220 may contain code for determining the capacity for the medical device using a device inventory. For example, medical device capacity code 220 may contain code configured to cause computer 101 to perform operation 320 described in reference to FIG. 3. Overcapacity code 230 may contain code for determining overcapacity when the patient caseload exceeds the capacity for the medical device. For example, overcapacity code 230 may contain code configured to cause computer 101 to perform operations 330-350 described in reference to FIG. 3. Alert code 240 may contain code for generating an alert based on determining overcapacity. For example, alert code 240 may be configured to cause computer 101 to perform operations 360-370 described in reference to FIG. 3.

Referring now to FIG. 3, a flow diagram of an example computer-implemented method 300 for managing unregulated device usage is depicted, according to embodiments.

Method 300 may be performed by any suitable computing system. As described above, method 300 may be performed by computer 101 by executing unregulated device usage module 200, however other computing systems are contemplated within the scope of the present disclosure.

At operation 310, the system determines a patient caseload for a medical device via EHR data for a clinical setting. The system may be communicatively coupled to the EHR system for the clinical setting. The system may access the EHR data from the EHR system via one or more application programming interfaces (APIs). For example, computer 101 may access EHR data for the clinical setting from remote server 104, public cloud 105, or private cloud 106.

The system may determine the patient caseload for the medical device in several ways. In some embodiments, the system may search the EHR data for references in active patient records indicating use of the medical device in their care. In some embodiments, the system may be configured to use inferential evidence to determine the use of a medical device. For example, an entry in a patient record of “IVAC. Penicillin 2cc” can be used to infer that an infusion pump is being used to deliver Penicillin to the patient. Thus, the system may be configured to determine that an infusion pump is being used on the patient when “IVAC. Penicillin 2cc” is found in the EHR data.

The system may be configured to work with various EHR systems including, but not limited to, Epic, Cerner, AllScripts, and Meditech. Similarly, embodiments of the present disclosure may work with various electronic healthcare standards including, but not limited to HL7, ADT, and FHIR. The system may analyze data from the standard data format in EHR. Additionally, in some embodiments, the system may analyze extensions to the standard data such as Z segments or narrative text of the medical professional. For example, when the EHR system uses the FHIR standard, the system may analyze the Observation and associated DeviceMetric for indications of medical device use. This may be a different medical device than the device that is associated with the DeviceMetric. For example, a DeviceMetric for a pulse oximenter may be included in the EHR data and narrative text in the DeviceMetric may indicate that a ventilator is being used to treat the patient. This indication may be explicit reference to a medical device or an implied us of a medical device.

At operation 320, the system determines the capacity of the medical device within the clinical setting. The system may access an electronic inventory of medical devices to determine the number of unique devices available. For example, the electronic inventory may be stored in remote database 130 and computer 101 may query the electronic inventory over WAN 102 via remote server 104. In some embodiments, the number of unique devices available may be determined by identifying whether the devices have a status of in service vs. out-of-service, e.g., for maintenance or cleaning. In some embodiments, a particular medical device may be approved for use with multiple patients at the same time. Thus, the capacity of the medical device may be the number of patients that a device is approved for use with multiplied by the number of unique devices available.

At operation 330, the system determines whether the medical device is over capacity based on the patient caseload and the capacity of the medical device in the clinical setting. That is, the system may determine whether the number of available medical devices in the clinical setting is sufficient to support the number of patients determined to be using the device. For example, if the medical device is only approved for use with a single patient, the system may determine overcapacity of the medical device if the patient caseload exceeds the number of unique devices. In some embodiments, the system may further perform a statistical analysis to determine, if the patient caseload exceeds the capacity of the medical device, what is the likelihood of overcapacity of the medical device vs. a delay in entering up to date information into either the EHR data or electronic inventory. Thus, a determination of overcapacity may be determined based on exceeding a threshold of the likelihood of overcapacity. If, at operation 330, the system determines that the medical device is not over capacity, the system may return to operation 310.

If, at operation 330, the system determines overcapacity of the medical device, the system may verify the device caseload at operation 340. This operation may be performed to verify that the electronic inventory is up to date. The system may alert a user to verify that medical devices that are marked as out-of-service are still out-of-service. The alert may be a communication to a user, for example, in a maintenance department of the clinical setting. The system may receive a response from the user indicating that one or more of the devices that were indicated as out-of-service in the electronic inventory have been returned to service, or confirmation that the devices are still out-of-service. For example, computer 101 may send a communication over WAN 102 to end user device 103, wherein end user device 103 is associated with a user involved in maintenance of the devices for the clinical setting, and receive a user response from end user device 103 via WAN 102. Additionally, the system may access the purchase history from the clinical setting's financial database used for order fulfillment to verify that the number of devices in the electronic inventory matches the number of devices that have been purchased. This may allow the system to determine that more medical devices are available than are in the electronic inventory.

At operation 350, the system may determine whether the medical device is over capacity based on verifying the device capacity. This may include adjusting the capacity of the medical device in response to determining, at operation 340, that one or more devices that were out-of-service have been returned to service. If a new capacity for the medical device has been determined, the system may compare the new capacity to the patient caseload. If at operation 350, the system determines that the medical device is no longer over capacity, the system may return to operation 310.

If, at operation 360, the system determines overcapacity of the medical device, the system may generate one or more device records in the electronic inventory to track one or more unregulated devices. For example, computer 101 may communicate with remote server 104, where remote server 104 maintains the device inventory in remote database 130, to generate a new record in the device inventory. In some embodiments, multiple records may be generated based on the difference between the device capacity and the patient caseload. The generated records may contain placeholder data such that a person user can enter the real data that applies to the device. This is because the specific device in use that is allowing for the overcapacity is unknown. Thus, in addition to creating the device record, the system may send a communication to one or more users to update the device record for the unregulated device. Alternatively, generating the device record may cause a notification to be communicated to a user. The generated device record may include a reference to the overcapacity medical device to assist user's in identifying the unregulated device.

At operation 370, the system may generate one or more alerts based on determining overcapacity. For example, computer 101 may generate one or more communications to end user devices 103 over WAN 102. In some embodiments, the system may send a communication to one or more clinical setting personnel to notify them of the overcapacity of the medical device and the potential use of unregulated devices. In some embodiments, the system may send communications to one or more clinical setting personnel instructing them to maintain a device record for any unregulated device usage such as, for example, recording information in the device record generated in operation 360.

After operation 370, the system may return to operation 310 and continue to monitor overcapacity of the medical device. If, the system determines, at operation 330 or operation 350, that the medical device in no longer over capacity, the system may generate another alert to one or more personnel in the clinical setting. This alert may instruct relevant personnel in the clinical setting to stop unregulated device usage. This may include removing unapproved devices or stopping the unapproved use of medical devices.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A computer-implemented method for tracking unregulated device usage in a clinical setting, the method comprising:
determining a patient caseload for a medical device via electronic health record (EHR) data for the clinical setting;
determining, by accessing an electronic inventory, a capacity for the medical device in the clinical setting;
determining overcapacity of the medical device based on the capacity and the patient caseload; and
generating an alert on unregulated device usage in response to determining the overcapacity of the medical device.

2. The method of claim 1, wherein the patient caseload is determined by inferring device usage for a patient based on EHR data for the patient.

3. The method of claim 1, wherein the patient caseload is determined by querying the EHR for patient records with a reference to the medical device.

4. The method of claim 1, further comprising generating a device record in the electronic inventory for an unregulated device.

5. The method of claim 4, wherein the generating an alert comprises communicating a notification to a user to enter information on an unregulated device into the device record.

6. The method of claim 1, further comprising:
in response to determining overcapacity, verifying an out-of-service status of a medical device, wherein the alerting is in response to verifying the out-of-service status.

7. The method of claim 1, further comprising:
in response to determining overcapacity, accessing a purchase history from a financial database used for order fulfillment; and
verifying that a number of devices in the electronic inventory matches a number of devices that have been purchased according to the purchase history.

8. A computer program product for tracking unregulated device usage in a clinical setting, the computer program product comprising a computer readable storage medium having program instructions embodied therewith, the program instructions executable by one or more processors to cause the one or more processors to perform operations comprising:
determining a patient caseload for a medical device via electronic health record (EHR) data for the clinical setting;
determining, by accessing an electronic inventory, a capacity for the medical device in the clinical setting;
determining overcapacity of the medical device based on the capacity and the patient caseload; and
generating an alert on unregulated device usage in response to determining the overcapacity of the medical device.

9. The computer program product of claim 8, wherein the patient caseload is determined by inferring device usage for a patient based on EHR data for the patient.

10. The computer program product of claim 8, wherein the patient caseload is determined by querying the EHR for patient records with a reference to the medical device.

11. The computer program product of claim 8, wherein the operations further comprise generating a device record in the electronic inventory for the unregulated device.

12. The computer program product of claim 11, wherein the generating an alert comprises communicating a notification to a user to enter information on an unregulated device into the device record.

13. The computer program product of claim 8, wherein the operations further comprise:
in response to determining overcapacity, verifying an out-of-service status of a medical device, wherein the alerting is in response to verifying the out-of-service status.

14. The computer program product of claim 8, wherein the operations further comprise:
in response to determining overcapacity, accessing a purchase history from a financial database used for order fulfillment; and
verifying that a number of devices in the electronic inventory matches a number of devices that have been purchased according to the purchase history.

15. A system for tracking unregulated device usage in a clinical setting, the system comprising:
one or more memories; and
one or more processors communicatively coupled to the one or more memories, the one or more processors configured to execute program instructions on the one or more memories to perform operations comprising:
determining a patient caseload for a medical device via electronic health record (EHR) data for the clinical setting;
determining, by accessing an electronic inventory, a capacity for the medical device in the clinical setting;
determining overcapacity of the medical device based on the capacity and the patient caseload; and
generating an alert on unregulated device usage in response to determining the overcapacity of the medical device.

16. The system of claim 15, wherein the patient caseload is determined by inferring device usage for a patient based on EHR data for the patient.

17. The system of claim 15, wherein the patient caseload is determined by querying the EHR for patient records with a reference to the medical device.

18. The system of claim 15, wherein the operations further comprise generating a device record in the electronic inventory for an unregulated device.

19. The system of claim 18, wherein the generating an alert comprises communicating a notification to a user to enter information on the unregulated device into the device record.

20. The system of claim 15, wherein the operations further comprise:
in response to determining overcapacity, verifying an out-of-service status of a medical device, wherein the alerting is in response to verifying the out-of-service status.
